(19) United States
(12) Patent Application Publication
(10) Pub. No.: US 2010/0181186 A1
(43) Pub. Date: Jul. 22, 2010

(54) Process for obtaining a purified hydrofluoroalkane

(75) Inventors: Ercan Uenveren, Hannover (DE); Eckhard Hausmann, Hannover (DE); Johannes Eicher, Sehnde (DE); Kerstin Eichholz, Langenhagen (DE)

Correspondence Address: Solvay;c/o B. Ortego - IAM-NAFTA, 3333 Richmond Avenue, Houston, TX, 77098-3099, US

(73) Assignee: SOLVAY FLUOR GMBH, Hannover (DE)

(21) Appl. No.: 12/668,779

(22) PCT Filed: Jul. 16, 2008
(86) PCT No.: PCT/EP08/59285

§ 371 Date: Jan. 12, 2010

(30) Foreign Application Priority Data
Jul. 20, 2007 (EP) 07112877.1

(51) Int. Cl. C01B 7/01; C07C 17/38; C07C 17/00; B01J 19/08

(52) U.S. Cl. 204/157.48; 570/262; 204/157.94; 422/186

(57) ABSTRACT

The invention consequently relates, in one aspect, to a process for obtaining a hydrofluoroalkane comprising at least two carbon atoms, which is purified of unsaturated organic impurities, according to which the hydrofluoroalkane containing organic impurities including (chloro)fluoro olefins is subjected to at least one purification treatment with bromine or BrCl, preferably in the presence of, an initiator. The process is suitable, for example, to purify 1,1,1,2-tetrafluoroethane. A further aspect concerns the application of LEDs or OLEDs to support chemical reactions of the gas-gas, liquid-liquid or gas-liquid type, and a respective reactor.

The present invention relates to a process for obtaining a purified hydrofluoroalkane chosen in particular from 1,1,1,2-tetrafluoroethane, 1,1,1,3,3-pentafluoropropane and 1,1,1,3,3-pentafluorobutane; and to the use of LEDs and OLEDs as radiation source in certain chemical processes.

Hydrofluoroalkanes such as 1,1,1,2-tetrafluoroethane, 1,1,1,3,3-pentafluoropropane and 1,1,1,3,3-pentafluorobutane may be used as replacements for (hydro)chlorofluoroalkanes, for example as blowing agents, as refrigerants or as solvents.

These hydrofluoroalkanes are typically manufactured by reacting a chloro or chlorofluoro precursor with hydrogen fluoride. The crude hydrofluoroalkanes obtained in this reaction often contain impurities such as unconverted reagents, hydrogen chloride and olefinic impurities, in particular chlorofluoro olefins containing 2, 3 or 4 carbon atoms.

Patent application WO-A-00/14040 describes a process for purifying 1,1,1,3,3-pentafluorobutane. According to this known process, it is possible to reduce the fluorotrichloroethylene content in 1,1,1,3,3-pentafluorobutane by ionic chlorination in the presence of FeCl₃, by hydrogenation in the presence of Pd/Rh on active charcoal or, in particular, by reaction with fluorine.

Patent application WO-A-97/37955 describes a process for purifying 1,1,1,3,3-pentafluoropropane of 1-chloro-3,3,3-trifluoropropene, in which a photochlorination initiated with UV light of wavelength from 300 to 400 nm is carried out.

According to patent application WO 2002/12153, hydrofluoroalkenes—that is to say olefins consisting solely of carbon, hydrogen and fluorine—are especially difficult to remove when they are present as impurity in a hydrofluoroalkane, in particular those comprising from 3 to 5 carbon atoms, most particularly those corresponding to the empirical formula C₄H₄F₄, present as impurity in particular in 1,1,1,3,3-pentafluorobutane. Said international patent application discloses several methods to purify hydrofluorocarbons, for example, by photochlorination of unsaturated impurities with light having wavelengths below 270 nm. Other methods relate to a reaction with HF, application of sorbents and certain methods of distillation.

On account of the very low chemical reactivity of some of the impurities, the removal by means of a chemical treatment of the hydrofluoroalkenes in hydrofluoroalkanes is liable to require prolonged treatment times that are undesirable in an industrial process for manufacturing hydrofluoroalkane. In an extreme case, it would not be possible to go below a certain hydrofluoroalkene content.

It was consequently desirable to have available a process for purifying hydrofluoroalkanes, in particular 1,1,1,2-tetrafluoroethane, 1,1,1,3,3-pentafluoropropane or 1,1,1,3,3-pentafluorobutane, which allows an effective reduction of the content of olefinic impurities and in particular of hydrofluoroalkenes while at the same time using technical means that are simple to implement.

The invention consequently relates to a process for obtaining a hydrofluoroalkane comprising at least two carbon atoms, which is purified of unsaturated organic impurities, according to which the hydrofluoroalkane containing organic impurities including (chloro)fluoro olefins is subjected to at least one purification treatment with bromine or BrCl. The term “hydrofluoroalkane” denotes saturated aliphatic compounds consisting of carbon atoms, hydrogen atoms and fluorine atoms. Thus, necessarily at least one hydrogen atom is present. According to the present invention, preferably hydrofluoroalkanes are purified which contain at least as many fluorine atoms as hydrogen atoms.

It is considered to be very surprising that bromine and BrCl add, at reasonable speed, to unsaturated impurities rather than to perform an undesired hydrogen-bromine exchange in the hydrofluoroalkane molecules which are to be purified.

The process according to the invention applies in particular to hydrofluoroalkanes comprising 2 to 6 carbon atoms. For example, 1,1,1,3,3,3-hexafluoropropane (HFC-236fa), 1,1,1,2,3,3,3-heptafluoropropane (HFC-227ea) and 1,1,1,2,3,4,4,5,5,5-decafluoropentane (HFC-43-10mee), 1,1,1,2-tetrafluoroethane, 1,1,1,3,3-pentafluoropropane or 1,1,1,3,3-pentafluorobutane, can be purified. The process is especially suitable for the purification of 1,1,1,2-tetrafluoroethane, 1,1,1,3,3-pentafluoropropane or 1,1,1,3,3-pentafluorobutane, and most particularly, for purifying 1,1,1,2-tetrafluoroethane.

It has been found surprisingly that the process according to the invention allows an effective reduction of the content of organic impurities in the hydrofluoroalkane. In particular, 1,1,1,2-tetrafluoroethane, 1,1,1,3,3-pentafluorobutane and 1,1,1,3,3-pentafluoropropane have physical and chemical stability under the conditions of the process according to the invention. The process according to the invention may be carried out easily.

The organic impurities whose content may be reduced by means of the process according to the invention generally comprise 2 to 6 carbon atoms, sometimes even more. If 1,1,1,2-tetrafluoroethane, 1,1,1,3,3-pentafluorobutane and 1,1,1,3,3-pentafluoropropane are treated, they generally comprise 2, 3 or 4 carbon atoms. The term “(chloro)fluoro olefins” denotes in the present invention olefins which are substituted by hydrogen atoms, chlorine atoms, and/or fluorine atoms with the proviso that at least one substituents is a chlorine or a fluorine atom. For example, the term includes chloro olefins, hydrochloro olefins, fluoro olefins, hydrofluoro olefines, chlorofluoro olefins and hydrochlorofluoro olefins. The hydrofluorocarbons to be purified may contain one or more of such (chloro)fluoroolefines. The impurities may comprise ethene, propene and/or butene substituted by at least one chlorine atom. They are in particular (chloro)fluoro olefins containing 2, 3 or 4 carbon atoms. Chlorofluoroethenes, chlorodifluoroethenes, for example, HFC-1122, chlorodifluoropropenes and chlorofluorobutenes are mentioned as examples of olefins which may be removed.

The process according to the invention is particularly suitable for effectively removing hydrofluoroalkenes and hydrofluorochloroalkenes which are present as contaminants in the hydrofluoroalkane to be purified. The process according to the invention allows an effective reduction of the content of olefinic impurities present especially in 1,1,1,2-tetrafluoroethane, 1,1,1,3,3-pentafluoropropane or 1,1,1,3,3-pentafluorobutane.

The treatment with bromine serves to brominate the olefinic impurities in the hydrofluoroalkane. These are notably (chloro)fluoro olefins containing 2, 3 or 4 carbon atoms or, in particular, the hydrofluoroalkenes, hydrofluorochloroalkenes and chloroalkenes mentioned above.

Specific examples of impurities which may be removed by the process of the present invention are 2,3,3,3-tetrafluoropropene, 1,1,3,3,3-pentafluoropropene, 1,2,3,3,3-pentafluoropropene, 3,3,3-trifluoropropene, 1,3,3,3-tetrafluoropropene, 1,1-difluorochloroethene (HCFC-1122), 1,2-difluorochloroethene (HCFC-1122a), trans-1-chloro-2-fluoroethene which may especially be present in 1,1,1,2-tetrafluoroethane, or monochlorotrifluorobutene isomers in 1,1,1,3,3-pentafluorobutane.

The reaction could be performed thermally, but it is preferably performed in the presence of an initiator. The initiator serves to decompose the bromine or BrCl molecules by cleavage.

Three variants of the process according to the present invention are preferred.

In a first variant of the process according to the invention, the initiator is a free-radical initiator selected from an organic or inorganic initiator compound.

To promote the mixing of the hydrofluoroalkane containing olefinic impurities with the initiator compound, the first variant of the first aspect of the process according to the invention is preferably carried out in the liquid phase.

According to the invention, the free-radical initiator is often an organic compound. Among the organic compounds that are usually used are peroxide or diazo compounds. Peroxide compounds are used in particular. Among these, the ones chosen more particularly are diacyl peroxides, peroxydicarbonates, alkyl peresters, peracetals, ketone peroxides, alkyl hydroperoxides and dialkyl peroxides. Diacyl peroxides or peroxydicarbonates are preferably selected. Excellent results have been obtained with dilauroyl peroxide, dibenzoyl peroxide or dicetyl peroxydicarbonate.

The free-radical initiator may also be an inorganic compound. In this case, it is often chosen from hydrogen peroxide, percarbonates such as, in particular, sodium percarbonate, and perborates such as sodium perborate.

The initiator compound is preferably selected from compounds with a half-life from 0.1 to 3 hours, preferably 0.5 to 1.5 hours and usually of about 1 hour at the temperature of the treatment with bromine.

The initiator compound is generally used in a proportion of at least about 10 ppm by weight relative to the hydrofluoroalkane containing olefinic impurities. Particularly, at least about 20 ppm by weight of initiator compound are used, even more particularly, at least about 30 ppm by weight. Most frequently, not more than about 10 000 ppm by weight of initiator compound are used relative to the hydrofluoroalkane containing olefinic impurities. Preferably, the amount of organic initiator compound does not exceed about 1 000 ppm by weight and even more preferably it does not exceed about 300 ppm by weight.

In the first variant of the process according to the invention, the bromine may be used in the gas phase or in the liquid phase. It is introduced in excess amounts relative to all of the olefinic impurities to be brominated in the hydrofluoroalkane containing olefinic impurities. Generally, the bromine is used in a proportion of more than 3 mol per mole of olefinic impurities and preferably at least about 4 mol per mole of olefinic impurities. Generally, it is not desirable to exceed about 40 mol of bromine per mole of olefinic impurities. It is preferable to limit the amount used so that virtually all of the bromine can react and is not found in unchanged form after the present purification treatment. Preferably, the amount does not exceed about 15 mol per mole of olefinic impurities, and even more preferably this ratio does not exceed about 12.

In the first variant of the process according to the invention, the treatment with bromine may be carried out over a wide temperature range. In particular, the treatment with bromine is carried out at a temperature of at least about 40° C. and even more particularly of at least about 60° C. Higher temperatures allow a faster conversion of the unsaturated compounds. However, this results in a correlative increase in pressure, of which account needs to be taken. Preferably, the treatment temperature does not exceed about 150° C. and even more preferably it does not exceed about 100° C. Excellent results have been obtained when the treatment with bromine is carried out in the regions of 60 to 100° C.

In the first variant of the process according to the invention, the treatment with bromine may be carried out at the autogenous pressure or a higher pressure generated, for example, by introducing an inert gas. In general, the treatment is carried out at a pressure which does not exceed about 5 MPa and preferably 2 MPa. Pressures from about 0.2 to about 2.0 MPa are very suitable for use.

These correlated conditions of high temperature and high pressure which are allowed for the treatment with bromine contribute towards the fast and effective removal of the olefinic impurities. In the first variant of the process according to the invention, the duration of the treatment with bromine may be from about 1 to about 120 minutes. Preferably, the duration of the treatment with chlorine is not more than about 60 minutes.

According to an advantageous embodiment of the first variant of the process according to the invention, the initiator compound is introduced into the hydrofluoroalkane containing olefinic impurities before the addition of bromine. In a preferred implementation variant of this embodiment of the invention, the bromine is introduced into the hydrofluoroalkane at a temperature close to the treatment temperature. In a particularly preferred implementation variant of this embodiment of the invention, the initiator compound is also introduced into the hydrofluoroalkane at a temperature close to the treatment temperature.

In a second variant of the process according to the invention, the initiator, which is a free-radical initiator, is an electromagnetic radiation. Light with a wavelength in the range from 320 to 540 nm is effective for the application of bromine. Here, light sources can be applied which emit radiation over the complete range from 320 nm to 540 nm, or which emit light only in one or more sub-ranges. Even light sources can be applied which emit radiation only in a narrow range and also light sources which emit radiation with a single wavelength such as laser sources. This does not exclude the applicability of light sources which emit radiation outside the range of 320 nm to 540 nm. It has to be noted, however, that here, a part of the energy radiated into the reaction mixture gets lost. Of course, if desired, different light sources can be combined. In view of the application of BrCl it shall be mentioned that radiation in a range between 300 nm and 520 nm is suitable. The treatment with bromine is preferred and will be explained in further detail.

For example, visible light, e.g. emitted by the sun or by common artificial light sources, can be applied as initiator. For example, light bulbs, halogen lamps or fluorescent lamps or fluorescent tubes can be used as light source. Alternatively, UV can be applied as initiator. Light with a wavelength in the range between 360 nm and 520 nm is very effective. Light with a wavelength in the range of 380 nm to 500 nm is especially effective. It is preferred to apply light sources the radiation of which includes at least fractions of radiation with wavelengths in the range of 380 to 500 nm. In one embodiment of this variant of the process according to the invention, the energy of the fraction of wavelengths higher than 360 nm is preferably higher than 5% of the total energy of the electromagnetic radiation. In another embodiment, the energy of the fraction of wavelengths shorter than 520 nm is at least 95% of the total energy of the electromagnetic radiation. Typical light sources suitable for the present invention are those that emit UV-A light (around 320 nm to 400 nm) and/or visible light. For example, low pressure, medium pressure or high pressure Hg lamps can be applied, for example, those doped with gallium iodide, cadmium iodide or thallium iodide. Fluorescent tubes are suitable, too. It has been found that light emitting diodes (LEDs) and organic light emitting diodes (OLEDs) are also very suitable to induce the initiation of bromine radicals. The advantage of LEDs is that they emit a very narrow spectrum of light. Thus, LEDs (or OLEDs) emitting light in an optimum range, close to the maximal extinction, can be selected. For example, diodes emitting light in the range of 390 nm to 460 nm are very suitable, because the maximum extinction coefficient of bromine is around 410 to 420 nm. LEDs (or OLEDs) emitting blue light are highly suitable. Those LEDs are commercially available.

It has been found, surprisingly, that this second variant of the process according to the invention is particularly effective for reducing to an acceptable level the amount of the (chloro)fluoroalkenes, for example, hydrofluoroalkenes, chlorofluoroalkenes and hydrochlorofluoroalkenes which may be contained in a hydrofluoroalkane, quickly and without substantial degradation of the hydrofluoroalkane. This variant of the process according to the invention allows the bromine to be used in the presence of a broad spectrum of wavelengths and allows a fast purification operation, an efficient destruction of the unsaturated impurities and an improved use of energy.

To protect lamps and burners, cooling may be applied. The separation between the lamp, lamps or burner and the reaction medium in which the purification reaction is carried out is generally achieved with a translucent material which allows the desired wavelengths to pass through. For example, radiation may be passed through quartz glass or borosilicate glass. While borosilicate glass is known to absorb radiation with a wavelength of lower than around 280 nm, this does not negatively affect the process of the present invention.

It is also possible to apply anti-corrosive coatings which are permeable for radiation (transparent), for example, protective paint or shrink tubes as described in U.S. Pat. No. 6,489,510. Protective coatings can be applied throughout the apparatus or on selected parts through which radiation is passing.

The photochemical purification process according to the invention is particularly suitable for purifying 1,1,1,2-tetrafluoroethane, 1,1,1,3,3-pentafluoropropane and 1,1,1,3,3-pentafluorobutane of hydrofluoroalkenes and hydrochlorofluoroalkenes.

This variant, like the other variants, of the process of the present invention can for example successfully be applied for the purification of 1,1,1,3,3-pentafluoropropane which comprises 1-chloro-3,3,3-trifluoropropene (R-1233zd) as an impurity.

It is also particularly suitable for purifying 1,1,1,3,3-pentafluorobutane of hydrofluoroalkenes of empirical formula C₄H₄F₄, in particular E-CF₃—CH═CF—CH₃, Z—CF₃—CH═CF—CH₃, E-CF₃—CH═CH—CH₂F, Z—CF₃—CH═CH—CH₂F, E-CF₃—CH₂—CH═CHF, Z—CH₃—CH₂—CH═CHF and/or CF₃—CH₂—CF═CH₂. The process is particularly suitable for purifying 1,1,1,3,3-pentafluorobutane of one or more hydrofluoroalkenes chosen from E-CF₃—CH═CF—CH₃, Z—CF₃—CH═CF—CH₃ and CF₃—CH₂—CF═CH₂. It also can be purified from C₄ClF₃H₄, tetrachloroethene or fluorotrichloroethene.

It is very particularly suitable for purifying 1,1,1,2-tetrafluoroethane (HFC-134a). For example, HFC-134a can be treated which comprises one of more of the following impurities: 1,2-difluoroethene (HFC-1132a/HFC-1132), trifluoroethene (HFC-1123), octafluoro-2-butene (FC-1318my), 2,3,3,3-tetrafluoropropene (HFC-1234yf), 1,1,3,3,3-pentafluoropropene (HFC-1225zc), 1,2,3,3,3-pentafluoropropene (HFC-1225ye), 3,3,3-trifluoropropene (HFC-1243zf), 1,3,3,3-tetrafluoropropene (HFC-1234ze), 1,1,1,4,4,4-hexafluoro-2-butene (HFC-1336m/z), 1,1-difluorochloroethene (HFC-1122), 1,2-difluorochloroethene (HFC-1122a), trans-1-chloro-2-fluoroethene (HCFC-1131), 1,1-dichloro-2,2-difluoroethene (CFC-1112a), trans-1,2-dichlorofluoroethene (HCFC-1121), trichloroethene (HCC-1120), chlorotrifluoroethene (CFC-1113), and vinylchloride (HCC-1140). Preferably, HFC-134a is treated which comprises one or more of the following impurities: 2,3,3,3-tetrafluoropropene (HFC-1234yf), 1,1,3,3,3-pentafluoropropene (HFC-1225zc), 1,2,3,3,3-pentafluoropropene (HFC-1225ye), 3,3,3-trifluoropropene (HFC-1243zf), 1,3,3,3-tetrafluoropropene (HFC-1234ze), 1,1,1,4,4,4-hexafluoro-2-butene (HFC-1336m/z), 1,1-difluorochloroethene (HFC-1122), 1,2-difluorochloroethene (HFC-1122a), trans-1-chloro-2-fluoroethene (HCFC-1131). Especially preferably, HFC-134a is treated according to the process of the present invention to remove or at least reduce the content of impurities selected from the group comprising 1,3,3,3-tetrafluoropropene (HFC-1234ze), 1,1-difluorochloroethene (HFC-1122), and trans-1-chloro-2-fluoroethene (HCFC-1131) and chlorotrifluoroethene.

The intensity of the electromagnetic radiation is generally at least 0.01 W h per kg of hydrofluoroalkane containing impurities, preferably at least 0.02 W·h·kg⁻¹ or even at least 0.05 W·h·kg⁻¹. The intensity of the electromagnetic radiation is generally not more than 5 W·h per kg of hydrofluoroalkane containing impurities and preferably not more than 3 W·h·kg⁻¹ or even not more than 2 W·h·kg⁻¹.

In the second variant of the process according to the invention, the bromine may be used in the gas phase or in the liquid phase. It is preferably used in the liquid phase.

The second variant of the first aspect of the process according to the invention may be carried out, for example, in a falling film photo reactor or in an immersed burner photo reactor.

In a first embodiment of the second variant of the first aspect of the process according to the invention, the bromine is introduced in stoechiometric or excess amounts relative to the entirety of the olefinic impurities to be brominated in the hydrofluoroalkane containing impurities. In this embodiment, the bromine is used in an amount of greater than or equal to about 1 mol per mole of olefinic impurities. The amount of bromine is, in this embodiment, generally less than or equal to about 10 mol of bromine per mole of olefinic impurities. Preferably, the amount does not exceed about 5 mol of bromine per mole of olefinic impurities and even more preferably this ratio does not exceed about 2.

In a second embodiment of the second variant of the process according to the invention, the bromine is introduced in amounts less than the entirety of the olefinic impurities to be brominated in the hydrofluoroalkane containing impurities. In this variant, the bromine is used in an amount less than 1 mole per mole of olefinic impurities, preferably in an amount of less than about 0.9 mol per mole of olefinic impurities. The amount of bromine is, in this embodiment, generally greater than or equal to about 0.01 mol of bromine per mole of olefinic impurities. Preferably, this amount is greater than or equal to about 0.1 mol of bromine per mole of olefinic impurities. An amount of greater than or equal to about 0.5 mol of bromine per mole of olefinic impurities is most particularly preferred.

In the second variant of the process according to the invention, the treatment with bromine is generally carried out at a temperature of greater than or equal to −30° C. At such low temperature, the photochemical reaction for the purification of 1,1,1,2-tetrafluoroethane can be performed pressureless in the liquid state. The temperature is often greater than or equal to 0° C. Preferably, the temperature is greater than or equal to about 10° C. In this variant, the treatment with bromine is generally carried out at a temperature of less than or equal to 150° C. The temperature is often less than or equal to 100° C. Preferably, the temperature is less than or equal to about 80° C. A very preferred range is 30° C. to 75° C.

In the second variant of the process according to the invention, the pressure at which the treatment with bromine is carried out is generally greater than or equal to about 1 bar. The pressure at which the treatment with bromine is carried out is generally less than or equal to about 40 bar. The expert skilled in the art knows that the vapor pressure of a specific compound at a certain temperature is the higher the lower the boiling point of that compound is. Accordingly, when 1,1,1,2-tetrafluoroethane is treated according to the present invention, the pressure is often equal to or higher than 5 bars (abs). It is often equal to or lower than 15 bars. If 1,1,1,3,3-pentafluorobutane is treated according to the present invention, the pressure is often equal to or higher than 1 bar (abs). It is often equal to or lower than 5 bars (abs). If 1,1,1,3,3-pentafluoropropane is treated according to the present invention, the pressure is often equal to or higher than 2 bars (abs). It is often equal to or lower than 10 bars (abs).

In the second variant of the process according to the invention, the duration of the treatment with bromine is variable. The duration is often dependent from the reaction conditions, from the speed of reaction of impurities with bromine and of course also from the desired degree of removal of impurities. For example, using lamps or burners with a high energy output or with radiation emitted close to the range of high extinction often allows for shorter treatments times needed to achieve a certain degree of purity compared to the use of lamps or burners with lower energy output or with radiation emitted less close to the range of high extinction. Often, the duration is greater than or equal to 0.1 minutes. The duration of the treatment with bromine is often greater than or equal to 1 minute. Preferably, the duration of the treatment with bromine is greater than or equal to 2 minutes. In the second variant of the first aspect of the process according to the invention, the duration of the treatment with bromine is generally less than or equal to 10 h. The duration of the treatment with bromine is often less than or equal to 5 h. Preferably, the duration of the treatment with bromine is less than or equal to about 1 h. In a particularly preferred manner, it does not exceed 30 minutes.

In a third variant of the first aspect of the invention, the initiator is an amount of a metal ion. The third variant is carried out preferably in the substantial absence of free-radical initiators. In particular it is preferably carried out in the substantial absence of electromagnetic radiation with a wavelength in the range of 320 nm to 540 nm. According to this variant, efficient elimination of hydrofluoroalkenes, chlorofluoroalkenes and hydrochlorofluoroalkenes such as mentioned above may be achieved, without substantial degradation of the desired hydrofluoroalkane. In this variant, no specific separation operation is required to separate the iniator from the hydrofluoroalkane. Alternatively, the initiator is separated easily by an optional distillation.

The metal ion is preferably a Lewis acid. It is preferably selected from ions of group IIIa, IVa and b, Va and b, VIb and VIII metals of the Periodic Table of Elements (IUPAC 1970). In a particularly suitable manner, it is selected from ions of iron, nickel, aluminium, boron, titanium, chromium, zirconium, tantalum, tin or antimony. Iron ions are particularly preferred. Iron halide compounds are very suitable, for example, FeCl₂, FeCl₃, FeBr₂ and FeBr₃.

The amount of metal ion present in the treatment with bromine is generally at most 10.000 ppm, often at most 5000 ppm and preferably at most 1000 ppm by weight relative to the hydrofluoroalkane containing organic impurities. The amount of metal ion is more frequently at most 100 ppm. The amount is preferably at most 50 ppm. An amount of metal ions of at most 30 ppm is particularly preferred. The amount of metal ion present in the treatment with bromine is generally at least 0.01 ppm by weight relative to the hydrofluoroalkane containing organic impurities. The amount of metal ion is more frequently at least 0.1 ppm. The amount is preferably at least 0.5 ppm.

The metal ion can be introduced into the reaction medium for example by addition of a suitable metal compound. In a particular embodiment, the treatment with bromine is carried out in a reactor made of a material containing a suitable metal as described above, under conditions sufficient to release at least a trace amount of metal ion.

In the third variant of the process according to the invention, the treatment with bromine is generally carried out at a temperature of greater than or equal to 0° C. The temperature is often greater than or equal to 20° C. Preferably, the temperature is greater than or equal to about 40° C. In this variant, the treatment with bromine is generally carried out at a temperature of less than or equal to 200° C. The temperature is often less than or equal to 150° C. Preferably, the temperature is less than or equal to about 100° C.

In the third variant of the first aspect of the process according to the invention, the duration of the treatment with bromine is generally greater than or equal to 1 h. The duration of the treatment with bromine is often greater than or equal to 3 h. In the third variant of the first aspect of the process according to the invention, the duration of the treatment with bromine is generally less than or equal to 20 h. Preferably, the duration of the treatment with bromine is less than or equal to about 10 h.

The suitable pressures in the third variant of the process according to the invention are the same as in the second variant of the process according to the invention.

In the third variant of the process according to the invention, the hydrofluoroalkane is suitably selected from the group consisting of 1,1,1,2-tetrafluoroethane, 1,1,1,2,3,3,3-heptafluoropropane, 1,1,1,3,3,3-hexafluoropropane, 1,1,1,3,3-pentafluoropropane and 1,1,1,3,3-pentafluorobutane. It is preferably selected from 1,1,1,2-tetrafluoroethane, 1,1,1,3,3-pentafluoropropane and 1,1,1,3,3-pentafluorobutane. Most preferably, the hydrofluoroalkane is 1,1,1,2-tetrafluoroethane or 1,1,1,3,3-pentafluorobutane.

In another embodiment, the third variant of the process according to the invention can also advantageously be used for the bromination of bulk chloro(fluoro) olefins such as described above or of fractions comprising a high amount of such chloro(fluoro) olefins.

The process according to the invention may be carried out in a batchwise, semi-continuous or continuous mode. A continuous mode is preferred.

In the process according to the invention, the bromination reactor and the distillation apparatus are preferably made of corrosion-resistant materials such as, in particular, alloys of the type such as MONEL, INCONEL or HASTELLOY.

In the process according to the invention, care is advantageously taken to ensure that the oxygen content in the bromine is less than 1000 ppm by volume and preferably that it does not exceed 50 ppm by volume. To do this, the hydrofluoroalkane containing olefinic impurities may first be deaerated by sparging with an inert gas, for example nitrogen. Often, the oxygen content in the starting material will fulfill these conditions, or the stating material will even be oxygen-free.

In the process according to the invention, the treatment with bromine is generally followed by a separation operation whose function is mainly to separate from the hydrofluoroalkane compounds with a higher boiling point, especially residual bromine, if comprised, and the formerly unsaturated impurities after they have been brominated. The separation operation is preferably a distillation.

The second variant, treatment with bromine (or BrCl) in the presence of radiation as initiator, is the preferred variant.

If desired, the process of the present invention can be performed in combination with other treatments known in the art. For example, it can be performed before or after one or more additional treatment steps selected from the group consisting of
(a) a treatment with chlorine or bromine in the presence of an initiator
(b) a reaction with hydrogen fluoride
(c) a distillation in which the purified hydrofluoroalkane is removed from the top of the distillation column or from the side
(d) an extractive distillation
(e) an adsorption onto a solid adsorbent
(f) a reaction with a compound containing oxygen, and
(g) a gas-phase reaction with a reagent capable of reacting with at least some of the organic impurities, with the exception of a reaction with elemental chlorine.

In the following, the additional treatment steps are explained in detail. By means of the additional steps, the hydrofluoroalkane can be pretreated to remove impurities, or to remove impurities which cannot be removed by a treatment with bromine, or with a too slow reaction speed. Alternatively, the hydrofluoroalkane already treated with bromine can be subjected to a subsequent additional treatment step, for example, to remove impurities which have not been removed or have not been removed to a satisfactory degree by the treatment with bromine, or to remove the formed bromination products.

In one additional treatment step, the hydrofluoroalkane is treated with elemental chlorine or once again with bromine in the presence of an initiator. The initiator serves to decompose the chlorine or bromine molecules by cleavage. The free-radical initiator is often an organic compound. Among the organic compounds that are usually used are peroxide or diazo compounds. Peroxide compounds are used in particular. The initiator may be visible light or UV light. The initiator may also be a metal ion which is preferably a Lewis acid. It is preferably selected from ions of group IIIa, IVa and b, Va and b, VIb and VIII metals of the Periodic Table of Elements (IUPAC 1970). In a particularly suitable manner, it is selected from ions of iron, nickel, aluminium, boron, titanium, chromium, zirconium, tantalum, tin or antimony. Iron ions are particularly preferred.

In another additional treatment step, the hydrofluoroalkane containing organic impurities is subjected to a reaction with hydrogen fluoride.

This makes it possible in particular to effectively reduce the content of organic impurities present in the hydrofluoroalkane by using hydrogen fluoride. The latter compound is among reagents used in a synthesis of a hydrofluoroalkane by hydrofluorination. The products of the conversion are saturated (hydro)fluoroalkanes which are toxicologically and environmentally more acceptable than olefinic or chlorofluoro organic impurities. In addition, for certain organic impurities, the reaction with hydrogen fluoride will lead to the formation of the desired hydrofluoroalkane, namely, in particular, 1,1,1,2-tetrafluoroethane, 1,1,1,3,3-pentafluoropropane or 1,1,1,3,3-pentafluorobutane. The additional step may be carried out readily by using technical means developed for reactions for the synthesis of hydrofluoroalkanes by hydrofluorination.

The organic impurities whose content may be reduced in particular by this additional step comprise at least one chlorine atom, such as chlorofluoroethylenes, chlorodifluoropropanes and chlorofluorobutanes or -butenes. They are in particular (chloro)fluoro olefins containing 2, 3 or 4 carbon atoms, such as chlorodifluoroethylene or monochlorotrifluorobutene isomers.

The additional hydrofluorination step is also particularly useful for the elimination of the (chloro)fluoroalkenes (which optionally may contain one or more hydrogen atoms) mentioned above. The reaction of the hydrofluoroalkene with hydrogen fluoride is preferably carried out in the presence of a fluorination catalyst. It may also be carried out in the absence of catalyst.

When the reaction of the (chloro)fluoroalkene (optionally containing one or more hydrogen atoms) with hydrogen fluoride is carried out in the presence of a catalyst, catalysts which can promote the addition of HF to an olefin and/or the replacement of a chlorine atom with a fluorine atom may be used. Among the catalysts which may be used, mention may be made of derivatives of metals chosen from the metals from groups IIIa, IVa and b, Va and b and VIb of the Periodic Table of the Elements (IUPAC, 1970) and mixtures thereof. Titanium, tantalum, molybdenum, boron, tin and antimony derivatives are more especially selected. Preferably, titanium or tin derivatives are used. Metal derivatives which may be mentioned are salts and more particularly halides. Preferably, the choice is made from chlorides, fluorides and chlorofluorides. Catalysts that are particularly preferred in the process for preparing the hydrofluoroalkane according to the invention are the chlorides, fluorides and chlorofluorides of titanium and of tin and mixtures thereof. Titanium tetrachloride and tin tetrachloride are particularly suitable for use.

The molar ratio between the hydrogen fluoride and the organic impurities present in the hydrofluoroalkane is generally at least 1 mol/mol. Preferably, the process is performed with a molar ratio of at least 1.5 mol/mol. The molar ratio between the hydrogen fluoride and the organic compound used generally does not exceed 1000 mol/mol. It is preferable for this molar ratio not to exceed 10 mol/mol. In this additional treatment step, a molar ratio between the hydrogen fluoride and the olefinic impurities of not more than 3 is often maintained.

The reaction with HF may be carried out in batchwise or continuous mode.

When the reaction is carried out in batchwise mode, the duration of the reaction of the hydrofluoroalkane containing organic impurities with hydrogen fluoride generally ranges from 10 min to 5 h. Preferably, this duration is at least 0.5 h. Advantageously, this duration is at least 1 h. In general, this duration does not exceed 4 h. Preferably, this duration does not exceed 2.5 h.

When the reaction is carried out in continuous mode, the residence time of the reagents in the reactor is generally at least 0.5 h. Usually it does not exceed 30 h. Typically it ranges from 5 to 25 h. Preferably, it ranges from 10 to 20 h. The expression “residence time of the reagents in the reactor” is intended to denote the ratio between the volume of the reaction medium and the flow rate by volume of the reaction medium at the reactor outlet.

In a first variant, which is preferred, the reaction of the hydrofluoroalkane containing organic impurities with hydrogen fluoride is carried out in the liquid phase. In this variant, the temperature at which the reaction of the hydrofluoroalkane containing organic impurities with hydrogen fluoride is carried out is generally at least 60° C. Preferably, the temperature is at least 80° C. In general, the temperature does not exceed 160° C. Preferably, it does not exceed 140° C.

In this variant, the pressure is chosen so as to keep the reaction medium in liquid form. The pressure used varies as a function of the temperature of the reaction medium. It is generally less than or equal to 40 bar. Preferably, it is less than or equal to 35 bar. In a particularly advantageous manner, the pressure is less than or equal to 25 bar. In general, the pressure is greater than or equal to 5 bar.

In a second variant, the treatment with HF is carried out in the gas phase. This variant is particularly suitable for purifying 1,1,1,2-tetrafluoroethane, 1,1,1,3,3-pentafluoropropane and 1,1,1,3,3-pentafluorobutane.

Specifically, 1,1,1,2-tetrafluoroethane, 1,1,1,3,3-pentafluoropropane and 1,1,1,3,3-pentafluorobutane show surprising thermal stability, which allows them to be purified in the gas phase.

In this second variant, a fluorination catalyst based on a metal oxide chosen from chromium oxide, zirconium oxide and aluminium oxide, and mixtures thereof, is often used. Often, the metal oxide has a specific surface area determined according to the BET method of at least 100 m²/g and preferably of at least 150 m²/g. Generally, this specific surface area is not more than 400 m²/g. The metal oxide is preferably amorphous.

In this second variant, the temperature of the reaction with hydrogen fluoride is generally at least 50° C. Preferably, the temperature is at least 100° C. Generally, the temperature is not more than 400° C. Preferably, the temperature is not more than 300° C.

The additional treatment with HF finds an advantageous application to the purification of a hydrofluoroalkane obtained by synthesis by hydrofluorination, in particular by hydrofluorination of a chloro(fluoro)carbon. In the latter case, it may be advantageous to reduce the hydrogen chloride content of the hydrofluoroalkane containing organic impurities prior to its use in the second aspect of the process according to the invention.

The treatment with HF is often followed by at least one treatment step intended to recover the hydrofluoroalkane. Examples of treatment steps which may be used are, inter alia, treatments which may be used to separate the residual hydrogen fluoride from the hydrofluoroalkane, such as, for example, adsorption onto a solid, for instance KF, NaF or alumina, washing with water, an extraction operation, a separation by means of a suitable membrane, an extractive distillation or a distillation.

According to another embodiment of the present invention, the hydrofluoroalkane containing organic impurities is additionally subjected to a distillation and the purified hydrofluoroalkane is removed from the top of the distillation column or from the side.

It has been found, surprisingly, that organic impurities present in the hydrofluoroalkane, in particular (hydro)(chloro)fluorocarbons comprising 2, 3 or 4 carbon atoms, do not have a tendency to form an azeotrope with the hydrofluoroalkane and can thus be separated. This treatment step may be carried out easily.

The organic impurities whose content may be reduced by the additional purification steps of the process according to the invention generally comprise 2, 3 or 4 carbon atoms. They are in particular brominated reaction products, often they are alkanes substituted by bromine and at least one of chlorine and fluorine; they may further comprise hydrogen. Usually, they contain 2, 3 or 4 carbon atoms. The distillation pressure is generally less than 40 bar absolute; often, it is less than 25 bars (abs). Generally, the distillation pressure is at least 0.5 bar. It is usually at least 1 bar. Preferably, it is at least 1.5 bar. The specific pressure applied is dependent from the basic product to be distilled. If the basic product is 1,1,1,3,3-pentafluorobutane, the pressure will preferably be in the lower region of the range given above. If the basic product is 1,1,1,2-tetrafluoroethane, the pressure will preferably be in the medium to upper region of the range given above.

In the present description of this additional step, any reference to the pressure corresponds to the absolute pressure measured at the top of the distillation column.

The temperature at which the distillation is carried out generally corresponds approximately to the boiling point of the hydrofluoroalkane at the chosen pressure.

When the hydrofluoroalkane is 1,1,1,3,3-pentafluorobutane, good results are obtained at a pressure of about 1.5 to 3 bar and a temperature of about 50 to 70° C., when the hydrofluoroalkane is 1,1,1,2-tetrafluoroethane, good results are obtained with a pressure of about 5 to 25 bars, and a temperature of about 20 to 75° C.

The distillation may be carried out in one or more distillation columns. Preferably, only one column will be used.

The distillation columns which may be used are known per se. It is possible to use, for example, conventional plate columns or “dual-flow” plate columns or columns with bulk or structured packing.

The number of theoretical plates in the distillation is generally at least 10. It is usually at least 15. A number of at least 20 give good results.

The feed of hydrofluoroalkane containing organic impurities in this additional treatment step is generally carried out at a level below 50% of the number of theoretical plates of the column, it being understood that the top of the column corresponds to 100% of the number of theoretical plates. This level is usually not more than 45% of the number of theoretical plates of the column. Generally, the feed is carried out at a level of at least 5% of the number of theoretical plates of the column. This level is usually at least 10% of the number of theoretical plates of the column.

If a side removal is carried out, it is generally carried out at the level which corresponds to at least 50% of the number of theoretical plates of the distillation. The side removal is generally carried out at the level which corresponds to not more than 80% of the number of theoretical plates of the distillation.

In this additional treatment step, the purified hydrofluoroalkane is generally removed in an amount of at least 50% of the feed. The amount is usually at least 70% of the feed. The amount is preferably at least 80% of the feed. Generally, the purified hydrofluoroalkane is removed in an amount of not more than 99% of the feed. The amount is usually not more than 97% of the feed. The amount is preferably not more than 95% of the feed.

The degree of molar reflux in the distillation is generally not more than 20. This degree is usually not more than 10. A degree of reflux of not more than 7 has given good results.

Another additional step which can be performed in the purification treatment according to the present invention is an extractive distillation. The extractive distillation is carried out in the presence of at least one extractant which is generally chosen from (hydro)chlorocarbons, (hydro)fluorocarbons, hydrochlorofluorocarbons, hydrocarbons, ketones, alcohols, ethers, esters, nitriles, hydrogen chloride and carbon dioxide.

Hydrofluorocarbons which may be used as extractants comprise typically from 1 to 6 carbon atoms, preferably from 1 to 4 carbon atoms. Preferred specific hydrofluorocarbon extractants are hydrofluoroalkane extractants chosen, for example, from difluoromethane, 1,1,1,2-tetrafluoroethane, 1,1,1-trifluoroethane, pentafluoroethane, 1,1,1,2,3,3,3-heptafluoropropane 1,1,1,3,3-pentafluoropropane and 1,1,1,3,3-pentafluorobutane. It is understood that the hydrofluorocarbon extractant in the fourth aspect of the process according to the invention is, in general, different from the hydrofluoroalkane containing organic impurities.

Other extractants which may be used are chosen, for example, from dichloromethane, perchloroethylene, n-pentane, n-hexane, methanol, ethanol, isopropanol, diethyl ether, acetone, 2-butanone, ethyl acetate and acetonitrile.

In another embodiment, the extractant is chosen from chlorinated precursors suitable for a synthesis of the hydrofluoroalkane by hydrofluorination or from chloro(fluoro) intermediates obtainable by hydrofluorination of a said chlorinated precursor, such as chlorofluoroethanes, chlorofluoropropanes and chlorofluorobutanes.

Preferably, the extractant is chosen from 1,1,1,3,3-pentachlorobutane, 1,1-dichloro-1,3,3-trifluorobutane, 1,3-dichloro-1,1,3-trifluorobutane, 3,3-dichloro-1,1,1-trifluorobutane, 1-chloro-1,3,3,3-tetrafluorobutane and 3-chloro-1,1,3,3-tetrafluorobutane or a mixture of these extractants.

The distillation is generally carried out at a pressure and a temperature which makes it possible essentially to avoid, where appropriate, the formation of azeotropes between the extractant and the hydrofluoroalkane.

The distillation may be performed in one or more distillation columns. Preferably, only one column will be used.

The distillation columns which may be used in the process according to the invention are known per se. It is possible to use, for example, conventional plate columns or “dual-flow” plate columns or columns with bulk or structured packing.

Still another alternative of an additional treatment step in the purification process of the present invention is an adsorption onto a solid adsorbent. The solid adsorbent may be chosen, for example, from aluminas, silicas, iron oxide compounds, zeolites and active charcoals. Such adsorbents are commercially available. The adsorbent is optionally activated prior to its use in the adsorption treatment. A heat treatment or a treatment intended to increase the Lewis acidity of the solid adsorbent is suitable. The preferred solid adsorbents are those which have undergone a treatment intended to increase their Lewis acidity, for example a washing with hydrochloric acid or with nitric acid.

The contact between the hydrofluoroalkane containing organic impurities and the solid adsorbent may be carried out according to various techniques. The process may be performed in a fluidized bed, but it is generally preferred to place the solid adsorbent in the form of a fixed bed of particles, through which is passed a flow of the hydrofluoroalkane containing organic impurities. This flow may be liquid or gaseous. In one variant, the adsorption is carried out in the gas phase.

When this additional process step is carried out in the gas phase, a contact time between the hydrofluoroalkane containing organic impurities and the solid adsorbent of at least 1 s is carried out. Preferably, the process is performed with a contact time of greater than 2 s. Good results have been obtained with a contact time of greater than or equal to 3 s. In principle, the process may be performed with a very long contact time, for example of several minutes. In practice, for reasons of efficiency, the process is generally performed with a contact time of less than 1 minute and preferably less than or equal to about 30 s.

When this additional process step is carried out in the liquid phase, a contact time between the hydrofluoroalkane containing organic impurities and the solid adsorbent of at least about 2 minutes is carried out. Preferably, the process is performed with a contact time of greater than about 5 minutes.

In principle, the process may be performed with a very long contact time, for example of 120 minutes. In practice, the process is generally performed with a contact time of less than 60 minutes and preferably less than or equal to about 30 minutes.

When the process step is carried out in a fixed bed, the contact time is defined as the ratio of the volume of the bed of adsorbent to the flow rate by volume of the stream of hydrofluoroalkane containing organic impurities. When the process step is carried out in a fluidized bed, the contact time is defined as the ratio of the volume of the tank containing the solid adsorbent to the flow rate by volume of the stream of hydrofluoroalkane containing organic impurities.

The solid adsorbent is used in the form of a powder of particles whose optimum particle size depends on the conditions under which the process is carried out. In general, a solid adsorbent whose particle diameter ranges from about 0.1 mm to 10 mm is selected. The process is preferably performed with particles with a diameter of less than or equal to 7 mm. In a particularly preferred manner, particles with a diameter of less than or equal to 5 mm are used. Moreover, it is preferred to use a solid adsorbent whose particles have a diameter of greater than or equal to 0.5 mm. The process is preferably performed with particles with a diameter of greater than or equal to 1 mm. In a particularly preferred manner, particles with a diameter of greater than or equal to 2 mm are used.

After the process, the solid adsorbent may be regenerated by heating at moderate temperature, for example 100 to 250° C., under a stream of gas, for example under nitrogen, or under reduced pressure. The solid adsorbent may also be regenerated by a treatment with oxygen.

Another alternative additional process step according to the invention concerns the purification treatment by reaction with a compound containing oxygen. It has been found that reagents containing oxygen react preferentially with the organic impurities present in the hydrofluoroalkane, in particular in 1,1,2-tetrafluoroethane or 1,1,1,3,3-pentafluorobutane and essentially without degrading the hydrofluoroalkane. The compound containing oxygen may be, for example, an oxygenated gas, an oxygenated acid, an organic or inorganic peroxide, a peroxide salt or a peracid. Specific examples of such compounds are chosen from oxygen, ozone, hydrogen peroxide, peracetic acid, potassium permanganate, sulphuric acid and sulphur trioxide.

In an embodiment of this additional process step, the reaction is carried out in the presence of a base and the compound containing oxygen is an alcohol. The base may be, for example, an alkali metal hydroxide such as sodium hydroxide or potassium hydroxide. The alcohol may be chosen, for example, from methanol, ethanol and isopropanol.

The reaction with the compound containing oxygen may be carried out in the presence or in the absence of an oxygenation catalyst. Oxygenation catalysts which may be used may be chosen, for example, from compounds and in particular from complexes containing platinum, manganese or titanium.

The reaction with the compound containing oxygen may be carried out in the gas phase or in the liquid phase. It is preferably carried out in the liquid phase. In this case, the reaction temperature is generally not more than 150° C. The temperature is more frequently not more than 120° C. Preferably, the temperature is not more than 100° C. The reaction temperature is generally at least −20° C. The temperature is more frequently at least 0° C. Preferably, the temperature is at least 20° C.

The reaction pressure is generally from 1 to 40 bar.

A further optional process step concerns a reaction in the gas phase with a reagent capable of reacting with at least some of the organic impurities. Here, the reagent may in principle be any reagent capable of reacting in the gas phase with at least some of the organic impurities present in the hydrofluoroalkane and in particular with the olefinic impurities. The reagent is advantageously chosen from hydrogen chloride, hydrogen, hydrogen fluoride, oxygen and ozone.

In a typical example, the reaction is a catalytic hydrogenation.

It has been found, surprisingly, that catalytic hydrogenation makes it possible to reduce the content of any impurity in particular in 1,1,1,2-tetrafluoroethane or 1,1,1,3,3-pentafluorobutane to a level close to, even less than, 5 mg/kg, while at the same time avoiding degradation of the hydrofluoroalkane.

Catalysts which may be used in the catalytic hydrogenation reaction in the gas phase according to the invention are, for example, catalysts containing a metal from group VIII of the Periodic Table of Elements (IUPAC, 1970) or a mixture of several metals, preferably supported on a support such as active charcoal, a fluorinated alumina or aluminium trifluoride. Specific examples of metals from group VIII are platinum, palladium and rhodium. Among these catalysts, a catalyst comprising palladium is preferred.

The metal content in the supported catalysts which may be used is generally at least 0.001% by weight. This content is usually at least 0.1% by weight. The metal content in the supported catalysts is generally not more than 20% by weight. This content is frequently not more than 10% by weight. A catalyst which is resistant with respect to the products which may be present during the catalytic hydrogenation, in particular hydrogen fluoride, is preferably chosen. Good results are obtained, for example, with a catalyst comprising palladium supported on active charcoal.

The molar ratio between the reagent and the organic impurities present in the hydrofluoroalkane is generally at least 1 mol/mol. Preferably, the process is performed with a molar ratio of at least 1.5 mol/mol. The molar ratio between the reagent and the organic impurities generally does not exceed 1000 mol/mol. It is preferable for this molar ratio not to exceed 10 mol/mol. In the seventh aspect of the process according to the invention, a molar ratio between the reagent and the olefinic impurities of not more than 3 is frequently maintained. However, when the reagent is hydrogen, good results are also obtained when a molar ratio between the hydrogen and the olefinic impurities of greater than or equal to 5 is maintained. The molar ratio between the hydrogen and the olefinic impurities is advantageously less than or equal to 20. Preferably, this ratio is less than or equal to 10.

The temperature of the gas-phase reaction is generally at least 50° C. This temperature is usually at least 70° C. Preferably, this temperature is greater than or equal to 100° C. Generally, the temperature of the gas-phase reaction is not more than 400° C. Preferably, this temperature is not more than 300° C. In a particularly preferred manner, this temperature is not more than 250° C. Even more preferably, this temperature is not more than 150° C.

In this additional step, it is often necessary to carry out an operation intended to place the hydrofluoroalkane containing the organic impurities into the gaseous form. This operation may comprise, for example, an evaporation. In one preferred variant, the operation comprises the removal, in the gaseous form, of a distillation fraction comprising hydrofluoroalkane and organic impurities, for the purpose of purifying it in the gas phase. The distillation fraction may be obtained by one or more distillations of crude hydrofluoroalkane comprising, in addition to organic impurities, possibly reagents arising as by-products or intermediates of the synthesis of the hydrofluoroalkane. The crude hydrofluoroalkane may in particular comprise hydrogen fluoride and/or hydrogen chloride, in particular when the hydrofluoroalkane is obtained by hydrofluorination. The hydrogen fluoride and/or hydrogen chloride content in the crude hydrofluoroalkane may be reduced by distillation, such that the distillation fraction has a low hydrogen fluoride and/or hydrogen chloride content.

This reduction of the hydrogen fluoride and/or hydrogen chloride content is particularly advantageous when a catalytic hydrogenation as described above is carried out. In this case, hydrofluoroalkane containing organic impurities and generally having an acidity of not more than 1000 mmol/kg, preferably of not more than 100 mmol/kg, is used in the purification treatment.

Good results are obtained with hydrofluoroalkane containing organic impurities that are essentially free of hydrogen fluoride and/or hydrogen chloride.

In the operation intended to place the hydrofluoroalkane containing the organic impurities into the gaseous form, care is generally taken to ensure that the temperature of the hydrofluoroalkane does not exceed the temperature of the gas-phase purification treatment.

The gas-phase purification reaction may be advantageously followed by one or more treatments intended to separate the hydrofluoroalkane from the products of reaction between the organic impurities and the reagent. A distillation is suitable as a treatment, in particular when the reagent is hydrogen.

In the process according to the invention, the purification treatment may be followed by one or more finishing steps intended, for example, to remove any residual acidity, in particular traces of hydrogen fluoride. A suitable finishing step for this purpose is, for example, an adsorption onto a solid such as alumina, KF, NaF or silica.

Other treatments which may be used are, for example, a washing with water, an extraction operation or a separation by means of a suitable membrane.

The process according to the invention applies to the purification of a hydrofluoroalkane containing olefinic impurities, prepared by any synthetic process, without a pretreatment being required. The process according to the invention also applies to the purification of a hydrofluoroalkane containing organic impurities, which consists essentially of hydrofluoroalkane and organic impurities. Typically, the hydrofluoroalkane to be purified contains not more than 10% by weight of organic impurities. This content of impurities may be not more than 5% by weight. It may even be not more than 1% by weight. The process according to the invention may even be applied to a hydrofluoroalkane containing not more than 0.1% by weight of organic impurities.

The process according to the invention finds an advantageous application in the purification of a hydrofluoroalkane obtained by hydrofluorination, in particular by hydrofluorination of a hydrochloro(fluoro)carbon.

It should be understood that the treatment with bromine according to the present invention can be combined with each additional purification treatments in order to optimize the benefits achieved by the process according to the invention. In a particular embodiment, the process according to the invention may be combined with 1, 2, 3 or 4 additional purification steps for removing organic impurities, including at least one purification treatment according to the invention. In particular the combinations allow for effective reduction of chloro(fluoro) olefin content with very low losses of desired hydrofluoroalcane.

In the following paragraph describing preferred combinations of purification treatments, the following abbreviations are used:
(a1) a treatment with bromine or BrCl according to the first variant of the process according to the invention (free-radical initiator selected from an organic or inorganic initiator compound);
(a2) a treatment with bromine or BrCl according to the second variant of the process according to the invention (electromagnetic radiation);
(a3) a treatment with bromine or BrCl according to the third variant of the process according to the invention (presence of a metal ion);
(b) a reaction with hydrogen fluoride;
(c) a distillation;
(d) an extractive distillation;
(e) an adsorption onto a solid adsorbent;
(f) a reaction with a compound containing oxygen;
(g) a gas-phase reaction, preferably a hydrogenation reaction;
(h) a photochlorination, photobromination or photochemical reaction with BrCl, for example, using exclusively UV light of a wavelength >280 nm
(i) a photolysis in the absence of chlorine
(j) a reaction with fluorine.

Suitable consecutive combinations include, amongst others (“+” meaning “followed by”

(a3)+(a1), (a3)+(a2), (a2+c), (c)+(a2)+(c), (a2)+(c), (a2)+(e), (a2)+(e)+(c), (a3)+(c), (a3)+(e), (a3)+(h), (b)+(a2), (a2)+(b)+(c), (c)+(a1), (c)+(a2), (d)+(a1), (d)+(a2), (f)+(a2), (g)+(a1), (g)+(a2), (i)+(a1), (i)+(a2), (j)+(a2),

Combinations (a3)+(a1), (a3)+(a2), (a3)+(c), (c)+(a2)+(c), (a2)+(c), (a2)+(e), (a2)+(e)+(c), (a2)+(e), (a3)+(e), (a3)+(h), (c)+(a1), (c)+(a2), (i)+(a1), (i)+(a2) are preferred.

It is understood that the aforementioned combinations are particularly well suited for the purification of 1,1,1,2-tetrafluoroethane, 1,1,1,3,3-pentafluoropropane and 1,1,1,3,3-pentafluorobutane.

It has to be noted that, under (h) above, a photochlorination is mentioned as possible additional treatment step together at least one treatment step according to the present invention. In this additional treatment step, light with a wavelength of >280 nm may be applied. It is also possible to apply light with a wavelength equal to or shorter than 280 nm. It even was found that light sources can be applied emitting radiation with a wavelength essentially around 254 nm. This is very surprising because radiation with such short wavelengths was expected to be ineffective in view of the absorption range of elemental chlorine. In fact, it was found that the treatment of hydrofluoroalkanes containing unsaturated impurities can be effected by reaction with elemental chlorine with electromagnetic radiation whereby the energy of the fraction of wavelengths shorter than 260 nm is at least 90% of the total energy of the electromagnetic radiation, can not only be applied as additional step together with a treatment using bromine or BrCl as described above; this treatment of hydrofluoroalkanes containing unsaturated impurities by reaction with elemental chlorine with electromagnetic radiation whereby the energy of the fraction of wavelengths shorter than 260 nm is at least 90%, preferably 100% of the total energy of the electromagnetic radiation, can even be applied as a single purification step (without treatment with bromine or BrCl as described above); or optionally combined with one or more of the steps (b)-(g) and (i) or (j) mentioned above. This aspect of the invention—to treat hydrofluoroalkanes containing unsaturated impurities with elemental chlorine—can even be performed with radiation whereby the energy of the fraction of wavelengths shorter than 260 nm is at least 90% of the total energy of the electromagnetic radiation. The term “hydrofluoroalkane” denotes those hydrofluoroalkanes denoted above; the preferred hydrofluoroalkanes are preferred in this aspect, too. The same applies for the unsaturated impurities. HFC-134a is the most preferred hydrofluoroalkane in this aspect. Preferably, at least one of the following impurities is contained in the HFC-134a and removed: 2,3,3,3-tetrafluoropropene (HFC-1234yf), 1,1,3,3,3-pentafluoropropene (HFC-1225zc), 1,2,3,3,3-pentafluoropropene (HFC-1225ye), 3,3,3-trifluoropropene (HFC-1243zf), 1,3,3,3-tetrafluoropropene (HFC-1234ze), 1,1,1,4,4,4-hexafluoro-2-butene (HFC-1336m/z), 1,1-difluorochloroethene (HFC-1122), 1,2-difluorochloroethene (HFC-1122a) and trans-1-chloro-2-fluoroethene (HCFC-1131).

Another aspect of the present invention is the application of electrically operated LEDs (light-emitting diodes) and electrically operated OLEDs (organic light-emitting diodes) as radiation source for performing specific photochemical reactions in gas-gas reactions, gas-liquid reactions, and liquid-liquid reactions. Thus, these diodes emit light when electric current is flowing.

Consequently, a further embodiment of the present invention is a process for performing a photochemical reaction of the gas-gas, liquid-liquid or gas-liquid type comprising the step of providing a reaction mixture from two or more starting reactants, initiating or supporting the reaction by delivering at least a part of the photochemical radiation by LEDs or OLEDs, and recovering a reaction product wherein the starting material includes organic compounds, and wherein the reaction is a photochemically supported chlorination, chlorobromination or bromination reaction, or a photoxidation reaction where the photoxidation is performed in the absence of a photosensibilizer, or in the presence of chlorine as photosensibilizer.

Preferably, the LEDs and OLEDs are applied in reactions between said inorganic diatomic molecules and organic reactants in gas-gas reactions, gas-liquid reactions, and liquid-liquid reactions in manufacturing processes or in purification processes.

An LED is a semiconductor device that emits incoherent narrow-spectrum light when electrically biased in the forward direction of the p-n junction. This effect is a form of electroluminescence. The color of the emitted radiation depends on the composition and condition of the semiconducting material used, and can be infrared, visible light or near-ultraviolet. LEDs are commercially available. For example, the following colors can be emitted:

Red and infrared: AlGaAs; green: AlGaP; yellow, green, orange, orange-red: AlGalnP; GaAsP: yellow, orangered and red; red, yellow and green: GaP; green, blue, white (if it has an AlGaN quantum barrier): GaN; near UV, bluish-green and blue: InGaN; blue: SiC as substrate; blue: Si (as substrate); blue: sapphire as substrate; blue: ZnSe; UV: diamond; near to far ultraviolet: AlN or AlGaN.

An OLED is an organic light-emitting diode. It comprises a film of organic compounds in the emissive electroluminescent layer. The layer usually contains a polymer substance that allows suitable organic compounds to be deposited in rows and columns on a flat layer by a simple printing process. The resulting matrix of pixels can emit light of different colors. A great range of colors can be produced.

Though they have a high efficiency, LEDs and OLEDs often have only comparably low energy output. Consequently, if desired, a multiple number of LEDs or OLEDs have to be applied together to achieve the desired output of energy.

In principle, LEDs and OLEDs can be applied in broad variety of the mentioned photochemical processes via gas-gas reactions, gas-liquid reactions, and liquid-liquid reactions, The process can for example comprise the steps of providing a reaction mixture from two or more starting reactants, initiating or supporting the reaction by delivering at least a part of the photochemical radiation by LEDs or OLEDs, and recovering reaction product. The recovery can include a purification step, often by distillation.

Such reactions include the hydrogen-halogen exchange, for example, to provide chlorinated or brominated compounds, chlorine, bromine or BrCl to unsaturated carbon-carbon bonds, for example, as described above for purification purposes, but of course, also with the intention to synthesize and manufacture compounds. They can for example generally be applied for converting unsaturated impurities comprised in saturated hydrofluorocarbons or perfluorocarbons into reaction products which are easier separable from the saturated hydrofluorocarbons or the perfluorocarbons which are to be purified. For example, fluoroethanes, fluoropropanes, fluorobutanes, fluoropentanes and higher homologues can be purified by removing unsaturated impurities in this manner. Among hydrofluorocarbons which can be purified, tetrafluoroethanes, e.g. 1,1,1,2-tetrafluoroethane, fluoropropanes, e.g pentafluoropropane, 1,1,1,3,3-pentafluoropropane, 1,1,1,2,3,3-hexafluoropropane or 1,1,1,3,3,3-hexafluoropropane, heptafluoropropanes, e.g. 1,1,1,2,3,3,3-heptafluoropropane, pentafluorobutanes, e.g. 1,1,1,3,3-pentafluorobutane shall be mentioned. The purification is preferably effected under photochemical contact with chlorine, BrCl, bromine or mixtures thereof. For example, 1,1,1,2-tetrafluoroethane can be purified from unsaturated impurities by adding chlorine, bromine or BrCl and applying LEDs or OLEDs as light source to support or promote the addition of the respective halogen to unsaturated impurities. For example, they can be applied as radiation source in the photochemically operated purification process described above which uses bromine or BrCl. It also can be used in a purification process wherein hydrofluoroalkanes are photochemically purified to remove (hydro)(chloro)fluoroalkenes.

The LEDs and OLEDs can also be applied in photochemical reactions involving oxygen and in the absence of any sensibilizer or in the presence of chlorine as additional sensibilizer (or “initiator”). If chlorine is used as sensibilizer, it is preferably the only sensibilizer used. Examples for reactions involving oxygen include the preparation of compounds comprising the C—(O) group from compounds with a CHCl group. In the frame of such reactions, carbonyl fluoride can be prepared from CH₂FCl, carboxylic acid chlorides from alkanes with a CHCl₂ group, and carboxylic acid fluorides from alkanes with a CHClF group.

For example, compounds of formula R′CFXC(O)Cl can be prepared as described on U.S. Pat. No. 5,545,298. X in this formula denotes fluorine or chlorine and R′ is fluorine or a perfluorinated saturated alkyl group with 1 to 10 carbon atoms.

Preferably, the reaction produces carboxylic acid chlorides of formula RC(O)Cl from respective chlorofluorocarbons of formula R—CHCl₂ wherein R is a C1 to C3 alkyl group substituted by at least one fluorine atom and optionally 1 or more Cl atoms, the CHCl₂ group is oxidized to the C(O)Cl group.

For example, chlorodifluoroacetyl chloride can be prepared by the photochemical reaction of CF₂ClCHCl₂ and oxygen; trifluoroacetyl chloride can be prepared from CF₃CHCl₂; and CF₃—CF₂C(O)Cl can be prepared from CF₃CF₂CHCl₂. In that patent, the reaction between the haloalkane and oxygen is performed pressureless the advantage being for example that glass reactors can be used. This reaction can also be performed at a pressure higher than ambient pressure, for example, at a pressure between 1 and 10 bars (abs.) or, if desired, at an even higher pressure. No chlorine is added.

U.S. Pat. No. 5,569,782 discloses a process for the preparation of polyfluorochloroalkylcarbonyl chlorides and perfluoroalkylcarbonyl chlorides, for example, perfluoropropionyl chloride, trifluoroacetyl chloride and chlorodifluoroacetyl chloride by photochemical oxidation of respective compounds with a CHCl₂ group which is converted to a C(O)Cl group. The reaction is performed in the presence of chlorine with light having a wavelength λ≧290 nm. This reaction can be performed pressureless.

Reactions to produce carboxylic acid chlorides and carboxylic acid fluorides according to the processes mentioned above using LEDs or OLEDs according to the present invention can also be performed under pressure.

For example, U.S. Pat. No. 3,883,407 discloses the preparation of trifluoroacetyl chloride by photochemical oxidation of CF₃CHCl₂ at a pressure up to 75 psig under applying UV light. According to the present invention, LEDs or OLEDs emitting UV light are applied.

U.S. Pat. No. 6,489,510 discloses a method for producing carboxylic acid fluorides of formula RCFXC(O)F. Here, X denotes fluorine or chlorine, and R represents fluorine or a linear or branched perfluorinated alkyl group with 1 to 9 carbon atoms. Advantageously, light of a wavelength of λ≧280 nm is applied. This process can be performed using LEDs or OLEDs as radiation source.

U.S. Pat. No. 5,663,543 discloses a method for producing a polyfluoropropionyl halide by photochemical oxidation of 3,3-dichloro-1,1,1,2,2-pentafluoropropane under formation of pentafluoropropionylchloride, or by photochemical oxidation of 1,3-dichloro-1,1,2,2,3-pentafluoropropane under formation of perfluoropropionylfluoride. The reaction is performed in the presence of chlorine and applying light with a wavelength longer than 280 nm. Also here, LEDs and OLEDs are applicable as radiation source according to the present invention.

International patent application WO 2005/085129 discloses a process for the photochemical preparation of carbonyl fluoride by photochemical oxidation of CHF₃ or CHF₂Cl with light of a wavelength equal to or longer than 280 nm in the presence of chlorine. LEDs and OLEDs are applicable radiation sources also in this process.

These reactions generally can be performed in the gas phase or in the liquid phase. Chlorination reactions, bromination reactions and chlorobromination reactions, especially those intended as purification processes, are preferably performed by providing a liquid starting material which is to be purified, and introducing gaseous halogen.

In the photooxidation reactions described above LEDs or OLEDs can be applied as light source. The photooxidation process is preferably performed as a gas-gas phase process; this means that the starting material, e.g. oxygen, CHFCl₂ or CF₃CHCl₂, is introduced into the reactor in gaseous state (this includes a vapor state), and any chlorine as sensibilizer is also introduced in gaseous form. If chlorine is involved, LEDs or OLEDs are selected such that they emit light preferably in the range between 280 and 400 nm, especially preferably in the range of 300 to 360 nm because this is the range of high extinction coefficients for chlorine. The preferred exclusion of light with wavelengths below around 280 nm has the additional advantage that some of the carboxylic acid halides produced absorb themselves light with a wavelength lower than about 280 nm. Absorbing light in that range may cause side reactions.

Photochemical oxidation of haloalkanes to form carboxylic acid halides or carbonyl fluoride and the application of LEDs or OLEDs in processes performed to for purification of compounds are preferred fields of application of the LEDs and OLEDs in the frame of the present invention. They also can be applied in other gas-gas, liquid-liquid and liquid-gas reactions. For example, they can be used as a source of radiation in the preparation of nitrogen-containing perfluoroalkylbromides as described in U.S. Pat. No. 5,486,275 by decarbonylation of respective nitrogen-containing perfluoroacyl bromides.

Gas-gas reactions are especially preferred.

Still another embodiment of the present invention is a reactor for photochemical reactions in the gas phase comprising LEDs or OLEDs as radiation-emitting source. The reactor for performing photochemically supported gas-gas reactions according to the invention comprises a reactor chamber for receiving a reaction mixture, one or more lines to supply fluids into the reactor, one or more lines to withdraw reaction fluid from the reactor, and at least one LED or OLED for applying radiation to the reaction mixture, a connection to a vacuum pump, and optionally, additional means common in reaction apparatus. Mention is made of means for mixing the reaction mixture, for example, mechanical or magnetic stirrers, means for heating, for example, external or internal heating elements, means for determining the temperature, for example, thermometers or thermo elements, means for cooling the reaction chamber or the LEDs or OLEDs, for example, cooling fingers and means for applying a vacuum or a vacuum and pressure, means for anticorrosive protection, for example, transparent paint or transparent shrink wraps on parts made of glass being in contact with a corrosive reaction medium (e.g. containing HF). The reactor is constructed such that a vacuum down to 0.1 bar (abs) and elevated pressure up to 15 bars (abs) can be applied without causing any damage of the reactor. Thus, the reactor is constructed vacuum-resistant and pressure-resistant. For example, it can be constructed of respectively thick glass walls or plastics or metal.

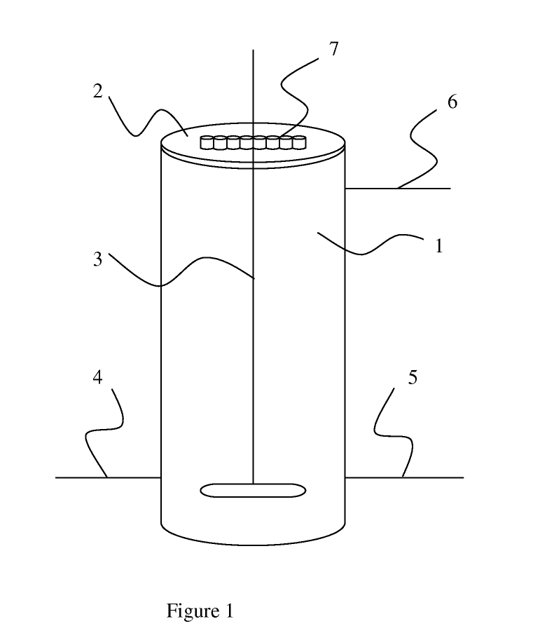

The rector according to the present invention is now explained in view of FIG. 1. The reactor comprises a reactor chamber 1 with pressure-resistant walls, bottom and top. The top 2 is a made from thick pressure-resistant, light-permeable borosilicate glass. A mechanical stirrer 3 serves to homogenize reaction mixture contained in reactor chamber 1. Fluid lines 4 and 5 serve to introduce gases into the reactor, while fluid line 6 serves to draw off reaction mixture. A group 7 of light-emitting diodes (LEDs) is arranged on the light-permeable top 2 so that the radiation of the LEDs is directed into the interior of the reactor. The reactor chamber may be equipped with sensors to determine physical conditions in the reactor, for example, temperature or pressure. FIG. 1 shows a very simple embodiment of the reactor according to the present invention. An immersion shaft photoreactor equipped with an irradiation unit comprising a plurality of LEDs instead of UV lamps is another suitable embodiment of the present invention. A line connectible to a vacuum pump is left out for the sake of simplicity. Alternatively, lines 4 and/or 5 can be connected to a valve, e.g. a 3-way valve which allows producing a vacuum in the reactor, e.g. for removing moisture or for performing reactions under reduced pressure.

The use of LEDS or OLEDs as described above has many advantages. For example, the LEDs and OLEDs can be selected such that light is emitted the wavelength of which corresponds to a range of maximal absorption. LEDs or OLEDs emitting light of different wavelength can be coupled and thus allow to construct an apparatus suitable to perform different reactions without the need to change light sources, or to perform effectively reaction steps where light of different wavelengths is needed.

The examples which follow are intended to illustrate the present invention without, however, limiting its scope.

### EXAMPLE 1

### Purification of 1,1,1,2-tetrafluoroethane

Photochemical reactor: a pressure-resistant cuvette which can be evacuated was used. It had a radius of 48 mm and a length of 25 mm. In the bottom, the cuvette comprised Schott Maxos borosilicate glass with a diameter of 63 mm and a thickness of 15 mm. The reactor content was irradiated through the borosilicate glass.

In examples 1.1 to 1.4, 1,1,1,2-tetrafluoroethane which was prepurified by distillation was treated, while in examples 1.5 to 1.7, raw product was used which was not prepurified in this manner. Prepurified and raw material differ especially in the content of impurities with higher boiling point (especially unsaturated impurities with 3 or 4 carbon atoms).

In all experiments, about 10 ml (0.007 g), 20 ml bromine vapor (0.014 g) or 40 ml bromine vapor (0.028 g) as indicated in tables 1 and II were introduced into the reactor, and then, 55.8 g of 1,1,1,2-tetrafluoroethane were added. The reactor content was thoroughly mixed whereby 1,1,1,2-tetrafluoroethane took a brown color resulting from the bromine. 1,1,1,2-tetrafluoroethane was essentially comprised in the cuvette in the liquid phase. The reaction mixture was then irradiated with the light sources indicated below. A part of the liquid phase was then transferred to a gas storage cylinder made from glass (covered with metal foil to protect against day light or lab light). The storage cylinder was then coupled to a respective analysis apparatus, and its content was analyzed by gas chromatography coupled with mass spectroscopy (GC-MS). It has to be noted that the GC-MS analysis performed was a qualitative one. The treated 1,1,1,2-tetrafluoroethane was analyzed to check the content of chlorotrifluoroethene (CFC-1113), tetrafluoropropene (HFC-1234) and chlorodifluoroethene (CFC-1122).
Example 1.1 was performed with day light, assisted by regular laboratory light. Example 1.1 was repeated using a gas storage cylinder in which the starting materials were reacted under the influence of day light and laboratory light. After the set time limit, the storage cylinder was covered with metal foil, coupled to the analysis apparatus, and the content was analyzed.
Examples 1.2.1 to 1.2.7 were performed using a UV-C light from Philips, model PL-S 9W.
Examples 1.3.1 to 1.3.9 were performed using a light bulb from Philips150 W with 2160 Lumen. This bulb emits white light in the form of a continuous spectrum of all colors, comparable to that of sun light.
Examples 1.4.1 and 1.4.2 were performed with 2×3 LEDs in series. The LEDs were purchased from Conrad Electronic (purchase number 187503) and are based on GaN, and emitted blue light around 470 nm, with a light intensity lᵥ of 4800 mcd.
Example 1.5 was performed with 38 of the LEDs described under examples 1.4.1 and 1.4.2.
Example 1.6.1 and 1.6.2 were performed with 4 LEDs in series. The LEDs of type LXHL-NRR8 were purchased from Conrad Electronic (purchase number b1716094-29) and emitted light at a wavelength of 455 nm called “royal blue” with a power of 1 W.
Examples 1.7.1 and 1.7.2 were performed with 38 LEDs as described under examples 1.4.1 and 1.4.2.
Examples 1.8.1 and 1.8.2 were performed with a UV high pressure lamp Sanolux HRC 300-280/E 27, available from Osram, was applied. It has a 300 W energy uptake and has a power 13.6 watts in the range of 315 to 400 nm (UV-A), and 3.0 watts in the UV-B range (around 280 to 320 nm).
Examples 1.9.1 and 1.9.2 were performed with a light emitting lamp “Ralutec long 18 W/71/2G11”. It has a power uptake of 18 watts and emits light in the range of 400 to 550 nm, with energy of 4.2 watts.

In the following table 1, data and results of the GC analysis of starting material (pretreated 1,1,1,2-tetrafluoroethane) and purified product of examples 1.1 to 1.4 are compiled. “X” denotes that the respective impurity was detected, “N” denotes that the respective impurity was below detection limit.

| | | Bromine | | | | |
|---|---|---|---|---|---|---|
| Light | | [mg/kg | Radiation | CFC- | HFC- | HCFC- |
| source | Example | 134a] | [min] | 1113 | 1234 | 1122 |
| — | Pretreated | — | — | X | X | X |
| | product | | | | | |
| Day + | 1.1 | 250 | 500 | N | N | N |
| lab light | | | | | | |
| UV-C | 1.2.1 | 250 | 2 | N | X | X |
| light | 1.2.2 | 250 | 7 | N | X | X |
| Philips | 1.2.3 | 250 | 17 | N | X | X |
| PL-S 9 W | 1.2.4 | 250 | 37 | N | X | N |
| | 1.2.5 | 250 | 97 | N | X | N |
| | 1.2.6 | 250 | 217 | N | N | N |
| | 1.2.7 | 500 | 30 | N | X | X |
| Light | 1.3.1 | 250 | 30 | N | N | N |
| bulb | 1.3.2 | 250 | 2 | N | X | X |
| Philips | 1.3.3 | 250 | 5 | N | X | N |
| 150 W | 1.3.4 | 250 | 10 | N | X | X |
| (2160 | 1.3.5 | 250 | 20 | N | X | N |
| lumen) | 1.3.6 | 125 | 30 | N | X | N |
| | 1.3.7 | 125 | 60 | N | X | N |
| | 1.3.8 | 500 | 15 | N | X | X |
| | 1.3.9 | 500 | 30 | N | N | N |
| 6 LEDs | 1.4.1 | 250 | 15 | N | X | N |
| | 1.4.2 | 250 | 30 | N | N | N |

Further tests were made with “raw” 1,1,1,2-tetrafluoroethane which was not prepurified by a destillation. The respective data are compiled in table 2:

TABLE 2
Examples performed with raw 1,1,1,2-tetrafluoroethane

| | | Bromine | | | | |
|---|---|---|---|---|---|---|
| Light | | [mg/kg | Radiation | CFC- | HFC- | HCFC- |
| source | Example | 134a] | [min] | 1113 | 1234 | 1122 |
| — | Raw | — | — | X | X | X |
| | product | | | | | |
| 38 LEDs | 1.5.1 | 500 | 90 | Not | Not | Not |
| | | | | determined | determined | determined |
| 4 LEDs | 1.6.1 | 250 | 5 | N | X | N |
| | 1.6.2 | 250 | 10 | N | N | N |
| 38 LEDs | 1.7.1 | 250 | 5 | N | N | N |
| | 1.7.2 | 250 | 10 | N | N | N |
| Sanolux | 1.8.1 | 250 | 5 | N | N | N |
| | 1.8.2 | 250 | 15 | N | N | N |
| Ralutec | 1.9.1 | 250 | 5 | N | N | N |
| 71 | 1.9.2 | 250 | 15 | N | N | N |

The treated 1,1,1,2-tetrafluoroethane can then be separated from brominated reaction products and residual bromine by known means, especially by distillation.

### EXAMPLE 1.2

### Purification of 1,1,1,3,3-pentafluoropropane

1,1,1,3,3-pentafluoropropane comprising 1-chloro-3,3,3-trifluoropropene is treated like described in the foregoing example by adding bromine and applying a light source. The molar ratio between bromine and 1-chloro-3,3,3-trifluoropropene is set to 1.2:1. After treatment, the reaction mixture is distilled to obtain purified 1,1,1,3,3-pentafluoropropane.

### EXAMPLE 1.3

### Purification of 1,1,1,3,3-pentafluorobutane

Example 1.2 is repeated, but 1,1,1,3,3-pentafluorobutane comprising C₄ClF₃H as unsaturated impurity is treated. Purified 1,1,1,3,3-pentafluorobutane is obtained after distillation.

### EXAMPLE 1.4

### Purification of 1,1,1,2-tetrafluoroethane using BrCl

Example 1 is repeated using BrCl as reactant to add to unsaturated impurities. The molar ratio of BrCl to the sum of unsaturated impurities is around 1.2:1. As light source, the light emitting lamp “Ralutec long 18 W/71/2G11” is applied. Purified 1,1,1,2-tetrafluoroethane is obtained by pressure distillation.

### EXAMPLE 1.5

### Purification of 1,1,1,2-tetrafluoroethane using Cl₂

Example 1 is repeated using Cl₂ as reactant to add to unsaturated impurities. The molar ratio of Cl₂ to the sum of unsaturated impurities is around 1.2:1. As light source, LEDs of type LXHL-NRR8 are applied. The chlorinated impurities can be separated from 1,1,1,2-tetrafluoroethane by pressure distillation.

### EXAMPLE 2

### Preparation of Trifluoroacetyl Chloride by Photochemical Oxidation of 1,1,1,-trifluoro-2,2-dichloroethane in the presence of chlorine

The process conditions as described by U.S. Pat. No. 5,569,782 in example 6 were applied. A mixture of pre-heated 1,1,1-trifluoro-2,2-dichloroethane and oxygen in a molar ratio of 1:1.2 is metered as a gas together with 38 mole-% of chlorine at an internal reactor temperature of 100° C. into a 400 ml immersed shaft photolysis reactor and simultaneously irradiated through Pyrex® glass using LEDs which emit UV light. LEDs 15-NJ available from Hooriba Jobin Yvon GmbH emitting light at 280 nm, or PSY-UVLED-280 available from Laser 2000 GmbH are examples of suitable UV sources. The pressure is maintained such that no condensation occurs in the reactor. The reaction mixture is then separated by usual methods, especially by pressure distillation.

### EXAMPLE 3

### Preparation of Carbonyl Fluoride by Photochemical Oxidation of CF₂HCl

As described, for example, in WO 2005/085129, an immersion shaft reactor with an internal volume of 580 ml is used. The cooling finger is made from Duran® glass which absorbs radiation with wavelengths below around 280 nm. Instead of the UV lamp described there, LEDs which emit UV, for example, those mentioned in example 2, are applied. Per hour, 0.5 moles of CHF₂Cl, 0.5 moles of oxygen and about 0.12 moles of chlorine are fed into the reactor. The reaction product comprises carbonyl fluoride, HCl, carbon dioxide and starting material which can be separated by pressure distillation, or more easily, by passing it through an ionic liquid as described in WO 2006/045518.

1- A process for obtaining a hydrofluoroalkane comprising at least two carbon atoms, which is purified of unsaturated organic impurities, according to which the hydrofluoroalkane containing organic impurities including (chloro)fluoro olefins is subjected to at least one purification treatment with bromine or BrCl.

2- The process according to claim 1, wherein the hydrofluoroalkane containing the olefinic impurities is subjected to a treatment with bromine in the presence of an initiator.

3- The process according to claim 2, wherein the hydrofluoroalkane is selected from the group consisting of 1,1,1,2-tetrafluoroethane, 1,1,1,3,3-pentafluoropropane and 1,1,1,3,3-pentafluorobutane.

4- The process according to claim 2, wherein the initiator is an organic initiator.

5- The process according to claim 2, wherein the initiator is an electromagnetic radiation comprising at least one fraction of wavelengths in the range of 320 nm to 540 nm.

6- The process according to claim 1, wherein the molar ratio between the bromine and the sum of the olefinic impurities present is from 1 to 10.

7- The process according to claim 1, wherein the molar ratio between the bromine and the sum of the olefinic impurities present is less than 1.

8- The process according to claim 1, wherein the initiator is a low amount of metal ion.

9- The process according to claim 1, wherein the treatment with bromine is carried out in the liquid phase.

10- The process according to claim 1, wherein the olefinic impurities comprise chlorofluoro olefins containing 2, 3 or 4 carbon atoms.

11- A process for performing a photochemical reaction of the gas-gas, liquid-liquid or gas-liquid type comprising the step of providing a reaction mixture from two or more starting reactants, initiating or supporting the reaction by delivering at least a part of the photochemical radiation by LEDs or OLEDs, and recovering a reaction product wherein the starting material includes organic compounds, and wherein the reaction is a photochemically supported chlorination, chlorobromination or bromination reaction, or a photoxidation reaction where the photoxidation is performed in the absence of a photosensibilizer, or in the presence of chlorine as photosensibilizer.

12- The process according to claim 11 wherein the reaction is a reaction to remove unsaturated impurities from haloalkanes.

13. The process according to claim 11 wherein the reaction is a reaction to produce carboxylic acid chlorides of formula RC(O)Cl from respective chlorofluorocarbons of formula R—CHCl₂ wherein R is a C1 to C3 alkyl group substituted by at least one fluorine atom and optionally 1 or more Cl atoms, and wherein the CHCl₂ group is oxidized to the C(O)Cl group.

14- A process for obtaining a hydrofluoroalkane comprising at least two carbon atoms, which is purified of unsaturated organic impurities, according to which the hydrofluoroalkane containing organic impurities including (chloro)fluoro olefins is subjected to at least one purification treatment with chlorine with electromagnetic radiation whereby the energy of the fraction of wavelengths shorter than 260 nm is at least 90% of the total energy of the electromagnetic radiation.

15- A reactor for performing photochemical reactions of the gas-gas type, comprising a reactor chamber for performing the photochemical reaction, further comprising one or more lines for delivery of gaseous starting material into the reactor, one or more lines for drawing off reaction mixture from the reactor and at least one LED and/or OLED to provide electromagnetic radiation to support the reaction between the starting materials, and comprising a line connectible to a vacuum pump, and wherein the reactor is vacuum-resistant and pressure-resistant.

16- The reactor according to claim 15 designed to perform photochemical reactions involving chlorine, equipped with at least one LED or OLED emitting light comprising at least one fraction of wavelengths in the range of 280 nm to 400 nm, or to perform reactions involving BrCl, equipped with at least one LED or OLED emitting light comprising at least one fraction of wavelengths in the range of 310 nm to 520 nm, or involving bromine, emitting light comprising at least one fraction of wavelengths in the range of 320 nm to 540 nm.

17- The process according to claim 4 wherein the organic initiator is selected from the group consisting of peroxide and diazo compounds.

18- The process according to claim 8 wherein the metal ion is selected from the group consisting of ions of group IIIa, IVa, IVb, Va, Vb, VIB, and VIII metals.

19- The process according to claim 12 wherein the reaction is a reaction to remove unsaturated impurities from fluoroalkanes.
